(19) United States
(12) Patent Application Publication
(10) Pub. No.: US 2026/0098857 A1
(43) Pub. Date: Apr. 9, 2026

(54) METHODS OF DETECTING BLOOD CLOTS/DEPOSITS ON BLOOD GAS ANALYZER SENSORS

(71) Applicant: Siemens Healthcare Diagnostics Inc., Tarrytown, NY (US)

(72) Inventor: Wei Zhang, Needham, MA (US)

(73) Assignee: Siemens Healthcare Diagnostics Inc., Tarrytown, NY (US)

(21) Appl. No.: 19/103,940

(22) PCT Filed: Aug. 15, 2023
(86) PCT No.: PCT/US2023/072224

§ 371 Date: Feb. 14, 2025

Related U.S. Application Data
(60) Provisional application No. 63/375,252, filed on Sep. 12, 2022.

(51) Int. Cl. G01N 33/49; G01N 27/416; G01N 35/10

(52) U.S. Cl. CPC G01N 33/4925; G01N 27/4163; G01N 27/4167; G01N 35/1016; G01N 2035/1018

(57) ABSTRACT

Electrochemical sensor modules for blood gas analyzers are disclosed that can detect the presence of a blood micro clot or deposit on a pH sensor. The electrochemical sensor modules include an integrated sensor chip containing a pH sensor and a bicarbonate (HCO₃) sensor. Kits and systems containing the electrochemical sensor modules are also disclosed, as well as methods of using the electrochemical sensor modules.

### CROSS REFERENCE TO RELATED APPLICATIONS/INCORPORATION BY REFERENCE STATEMENT

This application claims benefit under 35 USC § 119 (e) of U.S. Provisional Application No. 63/375,252, filed Sep. 12, 2022. The entire contents of the above-referenced patent application are hereby expressly incorporated herein by reference.

### STATEMENT REGARDING FEDERALLY SPONSORED RESEARCH OR DEVELOPMENT

Not applicable.

### BACKGROUND

Blood gas analyzers (“BGAs”) have been used for years in the medical industry to determine the presence and concentration of certain analytes which may be present in a patient's blood and/or blood sample. BGAs are routinely used by doctors, scientists, researchers, and medical-care professionals to determine the presence and/or concentrations of certain characteristics and/or analytes present in a patient's blood sample, including, without limitation: (1) blood gases (such as pH (acidity), carbon dioxide (measured as pCO₂-partial pressure of carbon dioxide), and/or oxygen (measured as pO₂-partial pressure of oxygen)); (2) electrolytes (such as sodium (Na⁺), potassium (K⁺), Calcium (Ca²⁺), and/or chloride (Cl⁻)); (3) metabolites (such as glucose, lactate, blood urea nitrogen (“BUN”), and/or creatinine); and/or co-oximetry concentration measurements (such as total hemoglobin (tHb), reduced hemoglobin/deoxyhemoglobin (HHb), oxyhemoglobin (O₂Hb), saturated oxygen (sO₂), carboxyhemoglobin (COHb), methemoglobin (MetHb), fetal hemoglobin (HbF), and/or bilirubin.

Blood clots or deposits occur very often in blood gas analyzers, and most of the blood clots/deposits result from the sample preparation procedure. However, even with implementation of careful preanalytical procedures, some micro clots and/or deposits still form and appear in the testing channel. In addition, micro clots can form on or around one or more sensors in the blood gas analyzer and thus interfere with response sensitivity (response slope) for the sensor(s).

The presence of micro clots/deposits can block the pathway of the blood gas analyzer, thereby reducing analyzer uptime and impacting sensor response with biased or erroneous results of critical blood gas parameters, such as (but not limited to) pH and pCO₂.

It is critical to identify the presence of a micro clot, as it can cause an erroneous response in (for example, but not by way of limitation) response slope, response offset, or recovery results. Once the micro clot is detected, the analyzer system can operate certain actions for removing micro clots/deposits, such as fluidic aspiration, or providing Question Result (QR) on the testing recovery.

In certain existing systems (such as, but not limited to, the RAPIDPoint® system; Siemens Healthineers, Tarrytown, NY), since the pH sensor is very sensitive to clot/deposit lodging on or around the cover membrane (and thereby interfering with local pH buffer capacity around the PVC surface (i.e., pH sensor)), these systems utilize two pH sensors (pH1 and pH2) in one module to identify a clot/deposit on the first pH sensor (pH1). If a micro clot is present on pH1, its local pH buffering capacity increases, and the sample's true pH value is not correctly responded. There is less chance for the second pH sensor to be coated with the same clot, and therefore the second pH sensor can still correctly respond with the sample's true pH value. To compare the pH recovery of both pH1 and pH2 sensors, the pH difference is used to identify the presence of a clot on pH1; this approach is referred to as a QSE (Quantum Slope Excursion) error. However, this correction method requires a single module to have space for two separate pH sensors, and thereby limits the use of the QSE approach in certain systems.

International Patent Application Publication No. WO2017/108647 discloses another method for detecting clots in a blood gas analyzer sensor channel. In this method, when a recovery result is suspected, additional special operation steps are enabled in which (1) a high-level analyte solution is filled in a channel first, then followed by (2) filling an exact volume of rinse/Call solution (normal level) in the channel (no flush), followed by an incubation for a period of time; in step (3), the channel is flushed with Rinse/Call. The signal difference between steps (2) and (3) is used to judge the presence or absence of clots, with which an empirical threshold is used. However, this method requires a special step to operate the sequence (therefore, uptime is limited). In addition, a special reagent with high concentration for all electrolytes is needed, which increases the risk to contaminate the next sample accuracy.

U.S. Pat. No. 11,169,141 discloses a method of detecting a clot by using at least two electrolyte sensors to compare their idle rinse reagent signal drift slopes; when the calculated relative idle mV drift slope (versus intact sensor idle drift slope) is larger than a specific threshold, it detects the presence of a clot on the target sensor. However, this method requires at least two sensors to be present for this comparison to obtain the “relative” signal drift slope for clot detection. Further, there is a presumption that the micro clot (or other deposit or bubble) only coats the target sensor but not the other sensors (such as baseline sensors); however, in a practical situation, the presence of a micro clot/deposit/bubble can indeed affect two or three sensors simultaneously. In addition, the method also relies on a second presumption that all sensors have the exact same response; however, it is possible that the response patterns can be different, especially at the end of use-life (near 30 days). This raises the risk that the pre-set threshold can give a false flag for a certain sensor.

U.S. Pat. No. 6,022,747 discloses a blood clot detector that includes an apparatus and method for detecting pipette tip obstructions prior to sample analysis. In particular, the blood clot detector includes a pressure transducer on an aspiration line to provide output voltage data to a microprocessor corresponding to the vacuum level during aspiration. However, the '747 patent does not provide any mechanism for detecting clots once the sample is delivered to the sensor module.

Therefore, there is a need in the art for new and improved devices and methods for detecting the presence of blood clots/deposits on blood gas analyzer sensors that overcome the disadvantages and defects of the prior art.

### BRIEF DESCRIPTIONS OF THE DRAWINGS

FIG. 1 demonstrates clot migration within a sensor channel by depicting a clot/deposit traveling in an electrochemical sensor module for pH and HCO₃. Panel A illustrates a clot before lodging on a pH sensor. Panel B illustrates a clot lodging on a pH sensor. The arrows depict the direction of fluidic flow.

FIG. 2A graphically depicts a pH slope for a pH sensor and a pH slope for a bicarbonate (HCO₃) sensor over a period of 20 days.

FIG. 2B graphically depicts a pH slope for a pH sensor and a pH slope for a bicarbonate (HCO₃) sensor during a three-day clot event.

FIG. 3 graphically depicts the use of a delta pH Slope (HCO₃-pH) for detecting a micro clot on a pH sensor according to one non-limiting embodiment of a method in accordance with the present disclosure.

FIGS. 4A and 4B graphically depict absolute mV signals of a pH sensor (FIG. 4A) and an HCO₃ sensor (FIG. 4B) during the clot period.

FIG. 5 graphically depicts a diagram of one non-limiting embodiment of a system constructed in accordance with the present disclosure.

FIG. 6 graphically depicts a flow chart of one non-limiting embodiment of a method in accordance with the present disclosure.

### DETAILED DESCRIPTION

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting in any way.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the terms “about” and “approximately” are used to indicate that a value includes the inherent variation of error for a composition/apparatus/device, the method being employed to determine the value, or the variation that exists among the study subjects. That is, the terms “about” and “approximately” and variations thereof are intended to include not only the exact value qualified by the term, but to also include some slight deviations therefrom, such as deviations caused by measuring error, manufacturing tolerances, wear and tear on components or structures, settling or precipitation of cells or particles out of suspension or solution, chemical or biological degradation of solutions over time, stress exerted on structures, and combinations thereof, for example. In particular, for example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a composition, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “patient” includes human and veterinary subjects. In certain embodiments, a patient is a mammal. In certain other embodiments, the patient is a human, including, but not limited to, infants, toddlers, children, young adults, adults, and elderly human populations. “Mammal” for purposes of treatment refers to any animal classified as a mammal, including human, domestic and farm animals, nonhuman primates, and zoo, sports, or pet animals, such as dogs, horses, cats, cows, etc.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

As used herein, the term “liquid sample,” “fluid sample,” and variations thereof will be understood to include any type of biological fluid sample that may be utilized in accordance with the present disclosure. Examples of biological fluid samples that may be utilized include, but are not limited to, whole blood or any portion thereof (i.e., plasma or serum), saliva, sputum, cerebrospinal fluid (CSF), intestinal fluid, intraperitoneal fluid, pleural fluid, cystic fluid, sweat, interstitial fluid, tears, mucus, urine, bladder wash, semen, combinations, and the like. In certain non-limiting embodiments, the typical liquid test sample utilized in accordance with the present disclosure is blood. The volume of the fluid sample utilized in accordance with the present disclosure can be from about 0.1 to about 300 microliters, or from about 0.5 to about 290 microliters, or from about 1 microliter to about 280 microliters, or from about 2 microliters to about 270 microliters, or from about 5 microliters to about 260 microliters, or from about 10 to about 260 microliters, or from about 15 microliters to about 250 microliters, or from about 20 microliters to about 250 microliters, or from about 30 microliters to about 240 microliters, or from about 40 microliters to about 230 microliters, or from about 50 microliters to about 220 microliters, or from about 60 microliters to about 210 microliters, or from about 70 microliters to about 200 microliters, or from about 80 microliters to about 190 microliters, or from about 90 microliters to about 180 microliters, or from about 100 microliters to about 170 microliters, or from about 110 microliters to about 160 microliters, or from about 120 microliters to about 150 microliters, or from about 130 microliters to about 140 microliters. In one non-limiting embodiment, the volume of the fluid sample is in a range of from about 100 microliters to about 200 microliters.

As used herein, the term “pO₂” will be understood to refer to the partial pressure of oxygen, that is, an amount of oxygen in a solution. “pO₂” may also be referred to as a level of oxygen dissolved in a solution.

As used herein, the term “pCO₂” will be understood to refer to the partial pressure of carbon dioxide, that is, an amount of carbon dioxide in a solution. “pCO₂” may also be referred to as a level of carbon dioxide dissolved in a solution.

The term “circuitry” as used herein includes, but is not limited to, analog and/or digital components, or one or more suitably programmed processors (e.g., microprocessors) and associated hardware and software or hardwired logic. The term “component,” as used in the context of circuitry, may include hardware, such as a processor (e.g., microprocessor), an application specific integrated circuit (ASIC), field programmable gate array (FPGA), a combination of hardware and software, and/or the like. In one non-limiting embodiment, the term circuitry refers to electronic circuitry and components related thereto necessary for the system module/analyte detection system to obtain quantitative measurements associated with a patient's liquid test sample, including, without limitation, a patient's whole blood sample, such as, by way of example only, spectrophotometric measurements related to the presence and/or concentrations of various analytes present in a patient's liquid test sample.

The term “software” as used herein may include one or more computer readable instructions that, when executed or initiated by a user, cause the system component and/or instrument (such as, by way of example only, a spectrophotometer within a blood gas analyzer) to perform a specified function (including, without limitation, the measurement of concentrations of various analytes present in a patient's liquid test sample). It should be understood that the algorithms described herein may be stored on one or more non-transient memory. Exemplary non-transient memory may include random access memory, read only memory, flash memory, and/or the like. Such non-transient memory may be electrically-based, optically-based, and/or the like.

Turning now to the various non-limiting embodiments of the present disclosure, devices, kit, systems, and methods are generally disclosed herein for the fast and efficient detection of micro clots/deposits on various sensors in a blood gas analyzer. In the devices, kits, systems, and methods, a pH sensor is utilized in combination with an HCO₃ sensor; while the HCO₃ sensor is traditionally used as a pCO₂ sensor, the present disclosure converts the HCO₃ sensor to respond to pH (when utilized with calibration reagents having the same pCO₂ concentration) and thereby serve as a secondary pH sensor. In a method of use, the pH slopes for the two sensors can be compared, and if the difference between the two slopes is above a threshold value, this indicates that a micro clot/deposit may be present.

Certain non-limiting embodiments are directed to an electrochemical sensor module for a blood gas analyzer. The electrochemical sensor module includes an integrated sensor chip that comprises a pH sensor and a bicarbonate (HCO₃) sensor. The electrochemical sensor module may include other sensors for detecting other analytes present in blood samples. For example, but not by way of limitation, the electrochemical sensor module may include one or more sensors for pO₂, sodium (Na⁺), potassium (K⁺), magnesium (Mg²⁺), calcium (Ca²⁺), chloride (Cl⁻), glucose, lactate, blood urea nitrogen (“BUN”), creatinine, and the like, as well as any combinations thereof.

The electrochemical sensor module may be shaped, sized, and configured in any manner that allows the electrochemical sensor module to function in accordance with the present disclosure. That is, the electrochemical sensor module may be provided with any shape, size, and configuration that allows the electrochemical sensor module to be disposed and permanently or releasably secured within a blood gas analyzer instrument for detection of one or more analytes present in a biological test sample.

The pH and HCO₃ sensors may be positioned on the electrochemical sensor module in any manner that allows the sensors to function in accordance with the present disclosure. In one non-limiting embodiment, the pH sensor is upstream of the HCO₃ sensor in the fluidic flow path to provide a pH measurement which is validated by the HCO₃ sensor.

The pH and HCO₃ sensors may be provided with any formulation known in the art or otherwise disclosed herein. For example, but not by way of limitation, each of the pH and HCO₃ sensors may comprise a cover membrane and an internal electrolyte layer. The formulations of the cover membranes for the sensors may be the same, while the internal electrolyte layers of the sensors are different. In certain particular (but non-limiting) embodiments, the sensors are potentiometric, solid-state sensors. pH and HCO₃ sensors are well known in the art, as are methods of producing same. Therefore, no further description thereof is deemed necessary.

Certain non-limiting embodiments are directed to a kit that comprises at least one of any of the electrochemical sensor modules disclosed or otherwise contemplated herein in combination with at least three calibration and/or quality control reagents. At least a first reagent and second reagent are for use in determining a pH slope of the pH sensor for the electrochemical sensor module, and the first and second reagents have pCO₂ concentrations that are substantially the same and pH values that are different. At least a third reagent is for use with the first reagent in determining a pH slope for the HCO₃/pCO₂ sensor for the electrochemical sensor module, and the first and third reagents have substantially the same pH values and different pCO₂ concentrations. In addition, the designation of the two reagents for use with the pH sensor and the two reagents for use with the HCO₃ sensor as “first,” “second,” and “third” reagents is solely for the purposes of illustrating the two different analyses that are performed, and the fact that two reagents are utilized for each.

The at least three calibration and/or quality control reagents can be provided with any formulations known in the art or otherwise contemplated herein that allow the calibration and/or quality control reagents to function in accordance with the present disclosure. In particular, any type of calibration reagent for use in the calibration and/or monitoring of the performance of blood gas analyzer systems, may be utilized in the formulation of the at least three calibration and/or quality control reagents. In addition to the calibration and/or quality control reagent(s), the at least three reagents may further comprise any other component necessary for functionality thereof, including, but not limited to, inorganic and/or organic salt(s), protein(s), catalyst(s), analyte(s), metabolite(s), and/or gas(es). Such types of calibration and/or quality control reagent(s) are well known in the art, and therefore no further discussion thereof is deemed necessary.

In particular (but non-limiting) embodiments, the cover membrane formulation of the HCO₃⁻ sensor is the same as the pH sensor, and the only difference between the pH sensor and the HCO₃⁻ sensor is the internal electrolyte composition. The pH sensor may have a strong buffered internal electrolyte solution layer to avoid impact from high pCO₂ from the sample side (while holding the free H+ ion concentration in the internal electrolyte phase unchanged, even with high pCO₂ diffusion from the sample side). The internal electrolyte solution layer of the HCO₃⁻ sensor may have minimal buffer so that any pCO₂ change from the sample side can be reflected from the internal electrolyte side via pCO₂ penetration through the cover membrane to change the pH of the internal electrolyte (IE) and be responded to from the inner side of the cover membrane (CO₂+H₂O←→H⁺+HCO₃⁻ in IE). In this manner, the pH sensor response signal is stabilized with substantially no CO₂ impact.

In certain particular (but non-limiting) embodiments, each of the at least three calibration and/or quality control reagents has a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, and the like, as well as a range of two of any of the above values (i.e., a range of from about 6.8 to about 7.4, etc.). Also, each of the at least three calibration and/or quality control reagents has a pCO₂ concentration of about 10 mmHg, about 15 mmHg, about 20 mmHg, about 25 mmHg, about 30 mmHg, about 35 mmHg, about 40 mmHg, about 45 mmHg, about 50 mmHg, about 55 mmHg, about 60 mmHg, about 65 mmHg, about 70 mmHg, about 75 mmHg, about 80 mmHg, about 85 mmHg, about 90 mmHg, about 95 mmHg, about 100 mmHg, and the like, as well as a range formed of two of any of the above values (i.e., a range of from about 35 to about 70 mmHg, etc.).

In certain particular (but non-limiting) embodiments, the first and third reagents have substantially the same pH, whereas the second reagent has a pH that is different than the pH of the other two reagents. For example, but not by way of limitation, the pH of the second reagent may be at least about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5 pH units higher or lower than the pH of the other two reagents. In a particular (but non-limiting) example, the second reagent has a pH that is at least about 0.5 pH units higher or lower than the other two reagents. In another particular (but non-limiting) example, the second reagent has a pH that is higher or lower than the pH of the other two reagents by a unit value that falls within a range between two of any of the above values (i.e., a range of from about 0.5 to about 2.5 pH units, a range of from about 0.3 to about 1 pH units, etc.).

In certain particular (but non-limiting) embodiments, the first and second reagents have substantially the same pCO₂ concentration, whereas the third reagent has a pCO₂ concentration that is different from the pCO₂ concentration of the other two reagents. For example, but not by way of limitation, the third reagent has a pCO₂ concentration that is at least about 0.25, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5 times than the pCO₂ concentration of the other two reagents. In a particular (but non-limiting) example, the third reagent has a pCO₂ concentration that is higher or lower than the pCO₂ concentration of the other two reagents by about 2.5× or less (such as, but not limited to, about 2× or less). In another particular (but non-limiting) example, the third reagent has a pCO₂ concentration that is higher or lower than the pCO₂ concentration of the other two reagents by a value that falls within a range between two of any of the above values (i.e., a range of from about 0.25× to about 2.5×, a range of from about 0.5× to about 2×, etc.).

In a particular (but non-limiting) embodiment, the first and third reagents have substantially the same pH that is in a range of from about 7.2 to about 7.6, and the second reagent has a pH that is below the pH of the other two reagents and is in a range of from about 6.6 to about 6.8. Alternatively, or in addition thereto, the first and second reagents have substantially the same pCO₂ concentration that is within a range of from about 20 mmHg to about 50 mmHg, and the third reagent has a pCO₂ concentration that is higher than the pCO₂ concentration of the other two reagents and is within a range of from about 60 mmHg to about 80 mmHg.

In a particular (but non-limiting) embodiment, each of the first and second reagents has a pCO₂ concentration of about 35 mmHg, and the first reagent has a pH of about 7.4, while the second reagent has a pH of about 6.8. Alternatively, or in addition thereto, each of the first and third reagents has a pH of about 7.4, and the first reagent has a pCO₂ concentration of about 35 mmHg, while the third reagent has a pCO₂ concentration of about 70 mmHg.

In addition to the electrochemical sensor modules(s) and calibration and/or quality control reagents, the kits disclosed herein may further contain other component(s) or reagent(s) for use when conducting any of the particular assays described or otherwise contemplated herein. For example, but not by way of limitation, the kit may further include at least one clot cleaning solution (such as, but not limited to, at least one clot cleaning solution (CCS) containing H₂O₂). The nature of additional reagent(s) present in the kits will depend upon the particular assay format, and identification thereof is well within the skill of one of ordinary skill in the art; therefore, no further description thereof is deemed necessary. Also, the compositions/reagents present in the kits may each be in separate containers/compartments, or various compositions/reagents can be combined in one or more containers/compartments, depending on the reactivity and stability of the compositions/reagents. For example (but not by way of limitation), the kit may further include positive and/or negative control reagents. In addition, the kit may further include a set of written instructions explaining how to use the kit. A kit of this nature can be used in any of the methods described or otherwise contemplated herein.

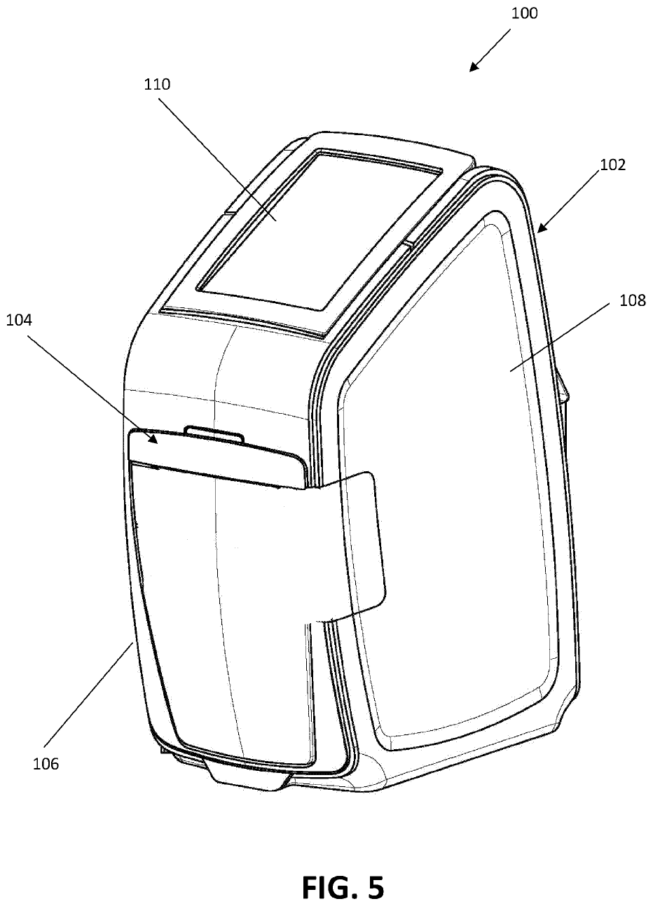

Certain non-limiting embodiments are directed to a system that includes one or more of any of the electrochemical sensor modules disclosed or otherwise contemplated herein in combination with a blood gas analyzer instrument; for example (but not by way of limitation), FIG. 5 illustrates a system 100 that includes a blood gas analyzer instrument 102 and an electrochemical sensor module 104. In certain particular (but non-limiting) embodiments the blood gas analyzer instrument 102 includes a housing 106 in which the electrochemical sensor module 104 is positioned and permanently or releasably secured. The instrument 102 may also include a processor 108 (or other form of evaluation unit) configured to predict the presence of a blood micro clot or deposit on the pH sensor of the electrochemical sensor module 104 based on a comparison of results obtained with the pH and HCO₃ sensors of the electrochemical sensor module 104. The blood gas analyzer instrument 102 may further include a reporting unit 110 for indicating when a blood micro clot or deposit is predicted to be present on the pH sensor of the electrochemical sensor module 104. For example (but not by way of limitation), the reporting unit may be any type of a renderer to provide a display, a printout, and/or an indication such as a visual (red or flashing light) and/or an audio indication(s), and may be configured to display a visual and/or auditory alert or indication (such as, but not limited to, a display and/or an auditory alert via a speaker) indicating the presence of the blood micro clot or deposit on the pH sensor. Alternatively, or in addition thereto, the reporting unit may be configured to provide an alert or indication of when a blood micro clot or deposit is predicted to not be present on the pH sensor.

In addition, the blood gas analyzer instrument may include one or more additional components involved in the performance of the methods described herein. For example, in certain particular (but non-limiting) embodiments, the blood gas analyzer instrument may include one or more sample inlets; a channel in which the electrochemical sensor module is positioned, wherein the channel forms a flow path through which the sample flows over the electrochemical sensor module (and any other sensor modules that are present); and an outlet through which the sample exits the flow path.

The blood gas analyzer instrument may also include compartments in which each of the first, second, and third calibration and/or quality control reagents are disposed or stored. The number of reagent compartments may correspond to the number of different reagents utilized. For example (but not by way of limitation), the blood gas analyzer instrument may contain at least three reagent-containing compartments for each of the first, second, and third calibration and/or quality control reagents.

In addition, in certain particular (but non-limiting) embodiments, the blood gas analyzer instrument further comprises at least one additional component for removing a micro clot or deposit from the pH sensor. One non-limiting example thereof is at least one clot cleaning solution containing H₂O₂, which may be stored in its own compartment.

Further additional components for blood gas analyzer instruments are widely known and used in the art, and therefore no further discussion thereof is deemed necessary.

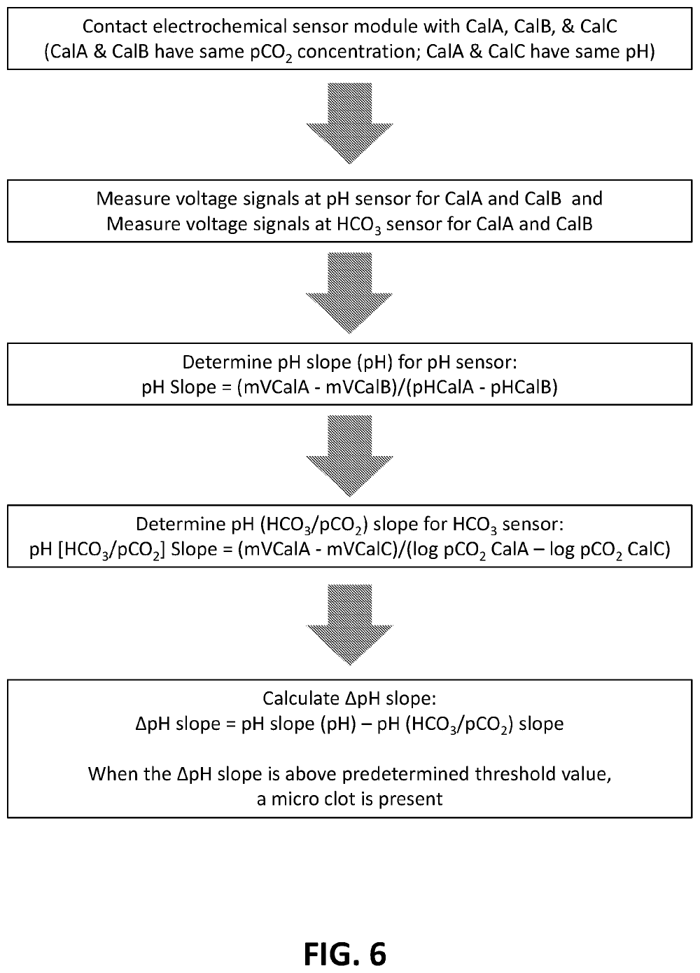

Certain non-limiting embodiments are directed to a method for detecting a micro clot or deposit on an electrochemical sensor module for a blood gas analyzer. The method includes the steps of: contacting any of the electrochemical sensor modules disclosed or otherwise contemplated herein with any of the first and second reagents disclosed or otherwise contemplated herein and measuring voltage signals to obtain first and second values, wherein the first and second reagents have pCO₂ concentrations that are substantially the same; contacting the electrochemical sensor module with any of the first and third reagents disclosed or otherwise contemplated herein and measuring voltage signals to obtain third and fourth values, wherein the first and third reagents have substantially the same pH; determining a pH slope (pH) for the pH sensor of the electrochemical sensor module based on the first and second values obtained with the at least first and second reagents; determining a pH (HCO₃/pCO₂) slope for the HCO₃ sensor of the electrochemical sensor module based on the third and fourth values obtained with the first and third reagents; calculating a ΔpH slope by subtracting the pH (HCO₃/pCO₂) slope from the pH slope (pH); and determining that a micro clot is present on the pH sensor if or when the ΔpH slope is above a predetermined threshold value. FIG. 6 contains a flow chart outlining the steps of the method.

The methods may include one or more additional steps, such as (but not limited to): generating a result indicating that the pH sensor result for a sample is questioned when the ΔpH slope is above the threshold value; and/or performing a clot removal step when the ΔpH slope is above the threshold value. The result generated may be an alert or indication, such as (but not limited to) a visual and/or auditory alert (as explained in more detail above).

As stated herein above, the use of the designations of “first,” “second,” and “third” reagents is solely for purposes of illustrating the values that are obtained. It should also be noted that the reagents utilized in accordance with the present disclosure are utilized sequentially; in other words, the reagents utilized contact the electrochemical sensor module separately. In addition, in certain particular (but non-limiting) embodiments, one or more wash fluids may contact the electrochemical sensor module in between calibration reagents.

In addition, while the methods described herein above utilize two calibration and/or quality control reagents for each slope determination (i.e., two-point calibration), it will be understood that three or more calibration and/or quality control reagents may be utilized for each slope calculation. That is, three or more calibration and/or quality control reagents may be utilized in determining the pH slope (pH) for the electrochemical sensor module, and/or three or more calibration and/or quality control reagents may be utilized in determining the pH (HCO₃/pCO₂) slope for the electrochemical sensor module. The only requirement is that all of the calibration and/or quality control reagents utilized to calculate the pH slope (pH) for the pH sensor must have substantially the same pCO₂ concentrations, while all of the calibration and/or quality control reagents utilized to calculate the pH (HCO₃/pCO₂) slope for the HCO₃ sensor must have substantially the same pH.

### Example

An Example is provided hereinbelow. However, the present disclosure is to be understood to not be limited in its application to the specific experimentation, results, and laboratory procedures disclosed herein after. Rather, the Example is simply provided as one of various embodiments and are meant to be exemplary, not exhaustive.

In this Example, an electrochemical sensor module is produced that contains an integrated sensor chip that includes at least the sensors of pH and HCO₃ (for pCO₂). In certain particular (but non-limiting) embodiments, the pH sensor is upstream of the HCO₃ sensor in the fluidic flow path. The cover membrane formulation of the HCO₃⁻ sensor is the same as the pH sensor; the only difference between the pH sensor and the HCO₃⁻ sensor is the internal electrolyte composition.

The pH sensor has a very strong buffered internal electrolyte solution layer to avoid impact from high pCO₂ from the sample side. The internal electrolyte solution layer of the HCO₃⁻ sensor has minimal buffer so that any pCO₂ change from the sample side can be reflected from the internal electrolyte side via pCO₂ penetration through the cover membrane to change the pH of the internal electrolyte (IE) and be responded to from the inner side of the cover membrane (CO₂+H₂O←→H⁺+HCO₃⁻ in IE). The pCO₂ response is obtained by the differential signal of co-existing pH and HCO₃ sensors. When the calibrators (i.e., calibration and/or quality control reagents) have identical pCO₂ levels, the HCO₃⁻ sensor is actually perfectly responding to pH, or in other words, measuring the concentration of hydrogen ions in the calibration solution (Table 1).

TABLE 1
pH and pCO₂ Composition in Calibration
Reagents for pH and pCO₂ Sensors

| | Cal A | Cal B | Cal C |
|---|---|---|---|
| | (First Reagent) | (Second Reagent) | (Third Reagent) |
| pH | 7.4 | 6.8 | 7.4 |
| pCO₂ mmHg | 35 | 35 | 70 |

The pH slope was calculated using Calibrators A and B (Cal A and Cal B), which have identical pCO₂ concentrations of 35 mmHg. The pH slope is calculated as:

$$pH Slope = ( mVCalA - mVCalB ) / ( phCalA - pHCalB ) \qquad ( 1 )$$

The HCO₃/pCO₂ slope was calculated using Calibrators A and C (Cal A and Cal C), which have identical pH concentrations of 7.4. The pH HCO₃/pCO₂ slope is calculated as:

$$pH [ {HCO}_{3} / {pCO}_{2} ] Slope = ( mVCalA - mVCalC ) / ( log pCO 2 CalA - log pCO 2 CalC ) \qquad ( 2 )$$

The HCO₃ sensor possesses the same pH sensitivity (slope) as the pH sensor when Cal A and Cal B were used for the pH slope calculation. For this purpose, the HCO₃ sensor can be regarded as a second pH sensor with a pCO₂ of 35 mmHg. Thus, there is no need for an additional pH sensor as in certain prior art methods.

Then the pH slopes are compared using the following formula:

$$ΔpH slope = pH slope ( pH ) - pH ( {HCO}_{3} / {pCO}_{2} ) slope \qquad ( 3 )$$

When the pH slopes from both sensors are compared as in equation (3), the difference should be constantly small (near zero) when no clot is lodged around the pH sensor.

When a micro clot is present on or around the pH sensor but not on or around the HCO₃ sensor, the local pH buffer environment around the occluded pH sensor is different from that of the intact HCO₃ sensor. The elevated local buffer capacity around the pH sensor makes the responding signal deviate from the expected mV value for either CalA or CalB. As such, the calculated slope of the pH sensor is suddenly reduced; however, the slope for the intact HCO₃ sensor does not change. Therefore, when a micro clot is present, the difference in slopes between the pH sensor and the HCO₃ sensor jumps to a significantly high level, and this is an obvious indicator of the presence of a micro clot on the pH sensor. The threshold of slope difference for micro clot presence can be pre-set based on experimental data from production sensors.

This Example provides a method for continuous or “on the fly” clot detection. HCO₃ is essentially regarded as a pH responding sensor with the use of two calibrators of the same pCO₂ level but different pH values (see, for example (but not by way of limitation), Cal A and Cal B in Table 1). This calculation on the HCO₃ sensor is different from the standard calibration scheme for the HCO₃⁻ sensor (pCO₂), which uses Cal A and Cal C for calculating HCO₃⁻/pCO₂ slope.

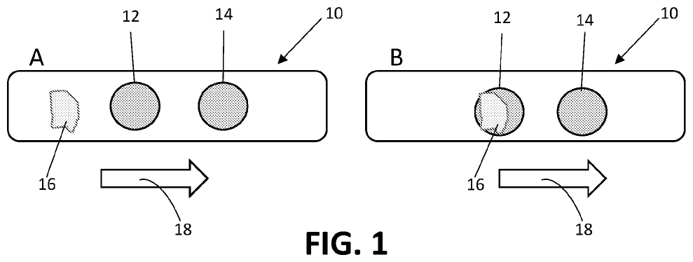

FIG. 1 illustrates migration of a micro clot 16 within a sensor channel 10 of an electrochemical sensor module having a pH sensor 12 and an HCO₃ sensor 14. Panel A depicts the micro clot 16 before lodging on the pH sensor 12, while Panel B depicts the micro clot 16 lodging on the pH sensor 12. As can be seen in FIG. 1, the micro clot 16 travels via fluidic flow (indicated by arrow 18) in the sensor channel 10 and is deposited on the pH sensor 12, which is upstream of the HCO₃ sensor 14. The presence of the micro clot 16 on the pH sensor 12 occludes the pH sensor 12 and interferes with the voltage signal measured by the pH sensor 12.

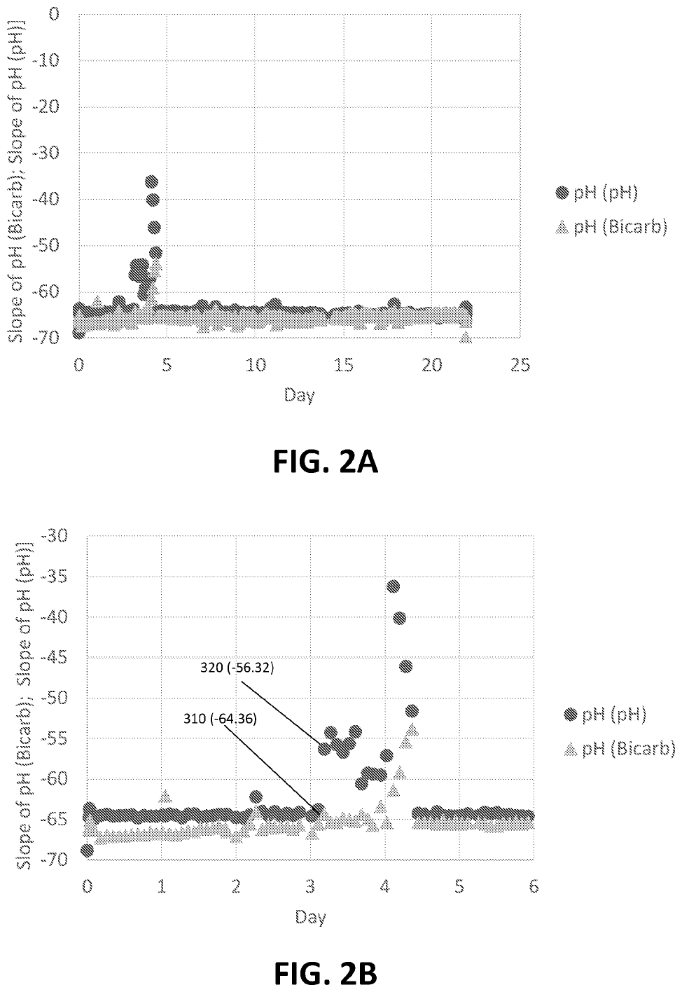

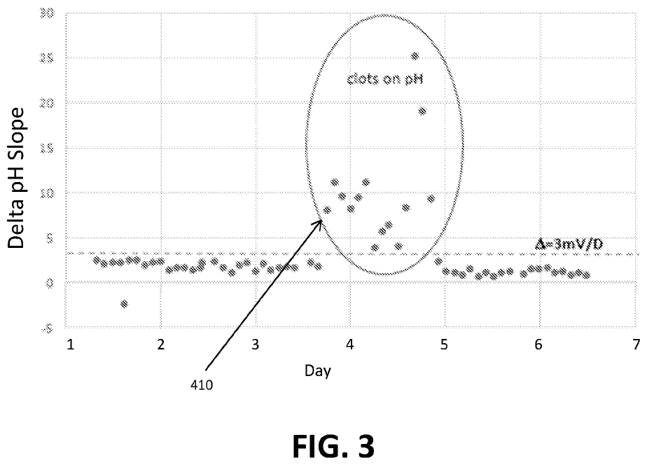

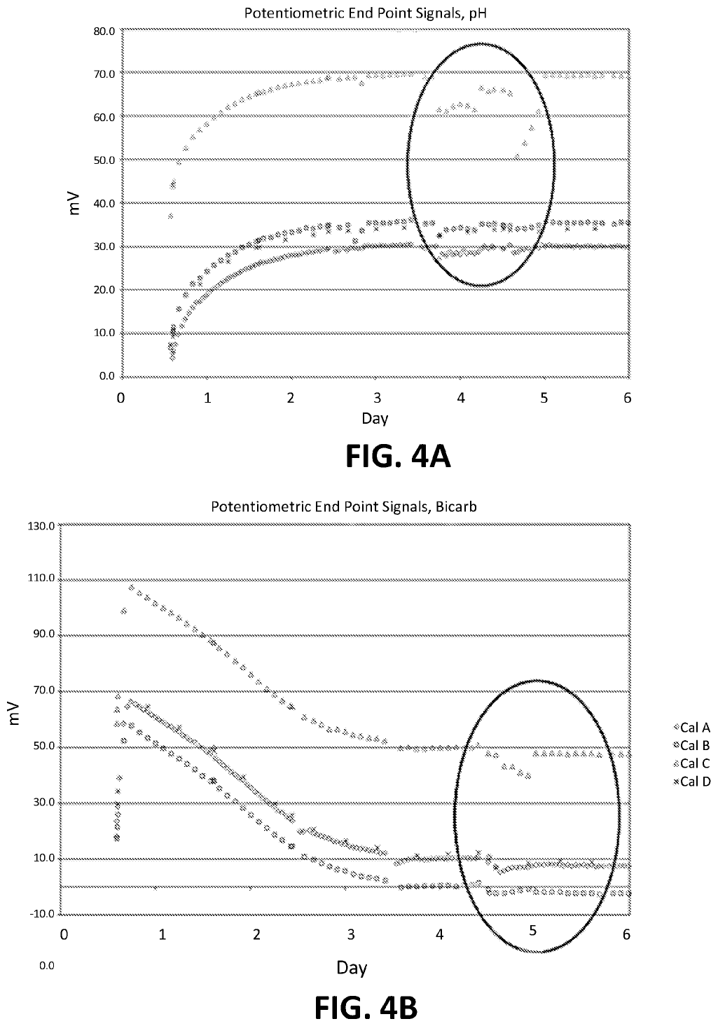

FIGS. 2A-2B, 3, and 4A-4B contain plots that indicate the functioning of a micro clot detection method in accordance with the present disclosure. The pH slope of the HCO₃ sensor is calculated and compared to the slope of the pH sensor (FIGS. 2A-2B). The delta pH slope is calculated as below (FIG. 3) and is the indicator of a micro clot on the surface of the pH sensor.

$$ΔpH slope = pH ( pH ) slope - pH ( {HCO}_{3} / {pCO}_{2} ) slope \qquad ( 3 )$$

In this Example (FIG. 3), the delta slope threshold is set as 3 mV/D. If or when the difference in pH slopes (i.e., the ΔpH slope) is consequently larger than the threshold value, then this result indicates that a micro clot is lodged on the pH sensor. When the delta slope is above the threshold, the analyzer subsequently generates a “Question Result” indication on the pH recovery (QR), and one or more additional actions required for clot removal can be triggered (e.g., fluidic aspiration, etc.).

Table 2 lists the calculated pH slope data for a pH sensor and an HCO₃ sensor of an electrochemical sensor module constructed in accordance with the present disclosure. It shows that at the time of 18:04, the slope of the pH sensor started to decrease from −63.80 to −56.32 mV/D (labeled 320 in FIG. 2B). Simultaneously, the pH slope of the HCO₃ sensor did not show such drop and was constant at approximately-65 mV/D (labeled 310 in FIG. 2B). The Delta pH between the pH and HCO₃ slopes jumped from approximately <2 mV/D to 8-10 mV/D magnitude (labeled 410 in FIG. 3). This is an indication or flag of micro clot presence on the pH sensor.

TABLE 2
pH Slopes of pH Sensor (Calculated from CalA and CalB) and
HCO₃ Sensor (Calculated From CalA and CalC) During Clot Event

| Day/Time | pH Slope pH | pH Slope HCO₃ | ΔpH Slope |
|---|---|---|---|
| Day 1 - 16:00 | −64.82 | −66.41 | 1.59 |
| Day 1 - 18:00 | −64.45 | −65.51 | 1.06 |
| Day 1 - 20:00 | −62.22 | −64.10 | 1.88 |
| Day 1 - 22:00 | −64.04 | −66.26 | 2.22 |
| Day 2 - 00:00 | −64.49 | −65.72 | 1.23 |
| Day 2 - 02:00 | −64.07 | −66.06 | 1.99 |
| Day 2 - 04:00 | −64.58 | −65.92 | 1.34 |
| Day 2 - 6:00 | −64.27 | −65.89 | 1.62 |
| Day 2 - 8:00 | −64.52 | −66.20 | 1.68 |
| Day 2 - 10:00 | −64.14 | −65.72 | 1.57 |
| Day 2 - 14:04 | −64.58 | −66.70 | 2.12 |
| Day 2 - 16:04 | −63.80 | −65.56 | 1.76 |
| Day 2 - 18:04 | −56.32 | −64.36 | 8.05 |
| Day 2 - 20:04 | −54.30 | −65.34 | 11.04 |
| Day 2 - 22:04 | −55.77 | −65.35 | 9.58 |
| Day 3 - 00:04 | −56.71 | −64.89 | 8.18 |
| Day 3 - 02:04 | −55.65 | −65.07 | 9.42 |
| Day 3 - 04:04 | −54.17 | −65.23 | 11.06 |
| Day 3 - 06:04 | −60.60 | −64.43 | 3.83 |
| Day 3 - 08:04 | −59.31 | −64.95 | 5.64 |
| Day 3 - 09:39 | −59.41 | −65.76 | 6.35 |
| Day 3 - 12:12 | −59.52 | −63.45 | 3.92 |
| Day 3 - 14:09 | −57.12 | −65.38 | 8.26 |
| Day 3 - 16:10 | −36.24 | −61.38 | 25.14 |
| Day 3 - 18:20 | −40.16 | −59.19 | 19.03 |

After a blood sample flows through tubing to reach the sensor cartridge, the size of most micro clots is in a range of from about 1 micrometer to several hundred micrometers in diameter. When the size of the micro clot is significantly smaller than the sensor surface size (which is approximately >1000 micrometers in diameter), the impact of the micro clot on sensor response can be neglected. However, when the size of the micro clot approaches (e.g., at least half the size) or covers at least about 50% of the sensor surface, the clot lodging on/around the sensor can be significant and should be identified.

When the front (or upstream) sensor (pH) is initially affected by a micro clot in the fluidic stream (i.e., when a micro clot covers at least a portion of the pH sensor), but the second (or downstream) sensor (HCO₃) is not affected, the system produces a flag (e.g., alert or indication), and clot cleaning/removal action (utilizing, for example, at least one clot cleaning solution (CCS), containing (for example, but not by way of limitation) H₂O₂) should be triggered immediately. After the cleaning/removal steps are performed, a response slope Cal sequence in accordance with the present disclosure is tested again to check for absence of the clot.

In summary, this Example describes a method of detecting the presence of a micro clot on a pH sensor within an integrated electrochemical sensor module. Using particular calibration agents as described, namely, two reagents for use with the pH sensor and two reagents for use with the HCO₃ sensor (e.g., as shown in Table 1), signals obtained from an on-board HCO₃ sensor during calibration are used to calculate a pH response slope for the HCO₃ sensor, and that pH (HCO₃/pCO₂) slope is compared to the pH slope from the pH sensor (pH slope (pH)). When a micro clot is present on the pH sensor, the difference between the pH slopes of the pH and HCO₃ sensors becomes suddenly elevated over the pre-set threshold, and this elevation is used as a micro clot indicator to trigger the Question Result (QR) and further clot removal operation(s) by the system.

### NON-LIMITING ILLUSTRATIVE EMBODIMENTS

The following is a list of non-limiting illustrative embodiments disclosed herein:

Illustrative embodiment 1. An electrochemical sensor module for a blood gas analyzer, the electrochemical sensor module comprising: an integrated sensor chip that comprises a pH sensor and a bicarbonate (HCO₃) sensor, wherein the pH sensor is configured to detect pH of a reagent, and wherein the HCO₃ sensor is configured to detect the pH of the reagent.

Illustrative embodiment 2. The electrochemical sensor module of illustrative embodiment 1, wherein the pH sensor is upstream of the HCO₃ sensor in the fluidic flow path.

Illustrative embodiment 3. The electrochemical sensor module of illustrative embodiment 1 or 2, wherein each of the pH and HCO₃ sensors comprises a cover membrane and an internal electrolyte layer, and wherein the formulations of the cover membranes for the sensors are the same, and the internal electrolyte layers of the sensors are different.

Illustrative embodiment 4. A kit, comprising: an electrochemical sensor module for a blood gas analyzer, the sensor module comprising an integrated sensor chip that comprises a pH sensor and a bicarbonate (HCO₃) sensor; at least a first calibration and/or quality control reagent and second calibration and/or quality control reagent configured to determine a pH slope for the pH sensor of the electrochemical sensor module, wherein the first and second calibration and/or quality control reagents have pCO₂ concentrations that are substantially the same; and at least a third calibration and/or quality control reagent configured for use with the first calibration and/or quality control reagent to determine a pH [HCO₃/pCO₂] slope for the electrochemical sensor module, wherein the first and third calibration and/or quality control reagents have substantially the same pH.

Illustrative embodiment 5. The kit of any one of illustrative embodiments 1-4, wherein the pH sensor of the electrochemical sensor module is upstream of the HCO₃ sensor in the fluidic flow path.

Illustrative embodiment 6. The kit of any one of illustrative embodiments 1-5, wherein each of the pH and HCO₃ sensors comprises a cover membrane and an internal electrolyte layer, and wherein the formulations of the cover membranes for the sensors are the same, and the internal electrolyte layers of the sensors are different.

Illustrative embodiment 7. The kit of any of illustrative embodiments claim 1-6, wherein each of the first, second, and third calibration and/or quality control reagents has a pH in a range of from about 6.8 to about 7.4 and a pCO2 concentration in a range of from about 35 to about 70 mmHg.

Illustrative embodiment 8. The kit of any of illustrative embodiments 1-7, wherein each of the first and second calibration and/or quality control reagents has a pCO2 concentration of about 35 mmHg, and the third calibration and/or quality control reagent has a pCO2 concentration of about 70 mmHg, and wherein each of the first and third calibration and/or quality control reagents have a pH of about 7.4, and the second calibration and/or quality control reagent has a pH of about 6.8.

Illustrative embodiment 9. A system, comprising: an electrochemical sensor module for a blood gas analyzer, the sensor module comprising an integrated sensor chip that comprises a pH sensor and a bicarbonate (HCO3) sensor; a blood gas analyzer instrument, comprising: a housing in which the electrochemical sensor module is positioned; a processor configured to predict the presence of a blood micro clot or deposit on the pH sensor of the electrochemical sensor module based on a comparison of results obtained with the pH and HCO3 sensors of the electrochemical sensor module; and a renderer configured to indicate when a blood micro clot or deposit is predicted to be present on the pH sensor.

Illustrative embodiment 10. The system of any one of illustrative embodiments 1-9, wherein the pH sensor of the electrochemical sensor module is upstream of the HCO3 sensor in the fluidic flow path.

Illustrative embodiment 11. The system of any one of illustrative embodiments 1-10, wherein each of the pH and HCO3 sensors comprises a cover membrane and an internal electrolyte layer, and wherein the formulations of the cover membranes for the sensors are the same, and the internal electrolyte layers of the sensors are different.

Illustrative embodiment 12. The system of any of illustrative embodiments 1-11, wherein the blood gas analyzer comprises: a first calibration and/or quality control reagent disposed in a first compartment; a second calibration and/or quality control reagent disposed in a second compartment; a third calibration and/or quality control reagent disposed in a third compartment; wherein the first and second calibration and/or quality control reagents have pCO2 concentrations that are substantially the same, and the first and third calibration and/or quality control reagents have substantially the same pH.

Illustrative embodiment 13. The system of any of illustrative embodiments 1-12, wherein the blood gas analyzer comprises at least one additional component for removing a clot or deposit from the pH sensor.

Illustrative embodiment 14. A method for detecting a micro clot or deposit on an electrochemical sensor module for a blood gas analyzer, wherein the electrochemical sensor comprises an integrated sensor chip that comprises a pH sensor and a bicarbonate (HCO₃) sensor, the method comprising the steps of: contacting the electrochemical sensor module with at least a first calibration and/or quality control reagent and second calibration and/or quality control reagent to obtain first and second values, wherein the first and second calibration and/or quality control reagents have pCO₂ concentrations that are substantially the same; contacting the electrochemical sensor module with at least the first calibration and/or quality control reagent and a third calibration and/or quality control reagent to obtain third and fourth values, wherein the first and third calibration and/or quality control reagents have substantially the same pH; determining a pH slope (pH) for the pH sensor of the electrochemical sensor module based on the first and second values obtained with the at least first and second calibration and/or quality control reagents; determining a pH (HCO₃/pCO₂) slope for the HCO₃ sensor of the electrochemical sensor module based on the third and fourth values obtained with the at least first and third calibration and/or quality control reagents; calculating a ΔpH slope by subtracting the pH (HCO₃/pCO₂) slope from the pH (pH) slope; and determining that a micro clot is present on the pH sensor when the ΔpH slope is above a threshold value.

Illustrative embodiment 15. The method of any one of illustrative embodiments 1-14, further comprising the steps of: generating a result indicating that the pH sensor result for a sample is questioned when the ΔpH slope is above the threshold value; and performing a clot removal step when the ΔpH slope is above the threshold value.

Illustrative embodiment 16. The method of any one of illustrative embodiments 1-15, wherein each of the first and second calibration and/or quality control reagents has a pCO₂ concentration of about 35 mmHg, and the third calibration and/or quality control reagent has a pCO₂ concentration of about 70 mmHg, and wherein each of the first and third calibration and/or quality control reagents have a pH of about 7.4, and the second calibration and/or quality control reagent has a pH of about 6.8.

Thus, in accordance with the present disclosure, there have been provided devices, kits, and systems, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. An electrochemical sensor module for a blood gas analyzer, the sensor module comprising:
an integrated sensor chip that comprises a pH sensor and a bicarbonate (HCO₃) sensor, wherein the pH sensor is configured to detect pH of a reagent, and wherein the HCO₃ sensor is configured to detect the pH of the reagent.

2. The electrochemical sensor module of claim 1, wherein the pH sensor is upstream of the HCO₃ sensor in the fluidic flow path.

3. The electrochemical sensor module of claim 1, wherein each of the pH and HCO₃ sensors comprises a cover membrane and an internal electrolyte layer, and wherein the formulations of the cover membranes for the sensors are the same, and the internal electrolyte layers of the sensors are different.

4. A kit, comprising:
an electrochemical sensor module for a blood gas analyzer, the sensor module comprising an integrated sensor chip that comprises a pH sensor and a bicarbonate (HCO₃) sensor;
at least a first calibration and/or quality control reagent and a second calibration and/or quality control reagent configured to determine a pH slope for the pH sensor of the electrochemical sensor module, wherein the first and second calibration and/or quality control reagents have pCO₂ concentrations that are substantially the same; and
at least a third calibration and/or quality control reagent, wherein the third calibration and/or quality control reagent is configured for use with the first calibration and/or quality control reagent to determine a pH [HCO₃⁻/pCO₂] slope for the bicarbonate sensor of the electrochemical sensor module, and wherein the first and third calibration and/or quality control reagents have substantially the same pH.

5. The kit of claim 4, wherein the pH sensor of the electrochemical sensor module is upstream of the HCO₃ sensor in the fluidic flow path.

6. The kit of claim 4, wherein each of the pH and HCO₃ sensors comprises a cover membrane and an internal electrolyte layer, and wherein the formulations of the cover membranes for the sensors are the same, and the internal electrolyte layers of the sensors are different.

7. The kit of claim 4, wherein each of the first, second, and third calibration and/or quality control reagents has a pH in a range of from about 6.8 to about 7.4 and a pCO₂ concentration in a range of from about 35 to about 70 mmHg.

8. The kit of claim 7, wherein each of the first and second calibration and/or quality control reagents has a pCO₂ concentration of about 35 mmHg, and the third calibration and/or quality control reagent has a pCO₂ concentration of about 70 mmHg, and wherein each of the first and third calibration and/or quality control reagents have a pH of about 7.4, and the second calibration and/or quality control reagent has a pH of about 6.8.

9. A system, comprising:
an electrochemical sensor module for a blood gas analyzer, the sensor module comprising an integrated sensor chip that comprises a pH sensor and a bicarbonate (HCO₃) sensor;
a blood gas analyzer instrument, comprising:
a housing in which the electrochemical sensor module is positioned;
a processor configured to predict the presence of a blood micro clot or deposit on the pH sensor of the electrochemical sensor module based on a comparison of results obtained with the pH and HCO₃ sensors of the electrochemical sensor module; and
a renderer configured to indicate when a blood micro clot or deposit is predicted to be present on the pH sensor.

10. The system of claim 9, wherein the pH sensor of the electrochemical sensor module is upstream of the HCO₃ sensor in the fluidic flow path.

11. The system of claim 9, wherein each of the pH and HCO₃ sensors comprises a cover membrane and an internal electrolyte layer, and wherein the formulations of the cover membranes for the sensors are the same, and the internal electrolyte layers of the sensors are different.

12. The system of claim 9, wherein the blood gas analyzer comprises:
a first calibration and/or quality control reagent disposed in a first compartment;
a second calibration and/or quality control reagent disposed in a second compartment;
a third calibration and/or quality control reagent disposed in a third compartment;
wherein the first and second calibration and/or quality control reagents have pCO₂ concentrations that are substantially the same, and the first and third calibration and/or quality control reagents have substantially the same pH.

13. The system of claim 12, wherein the blood gas analyzer comprises at least one additional component for removing a clot or deposit from the pH sensor.

14. A method for detecting a micro clot or deposit on an electrochemical sensor module for a blood gas analyzer, wherein the electrochemical sensor comprises an integrated sensor chip that comprises a pH sensor and a bicarbonate (HCO₃) sensor, the method comprising the steps of:
contacting the electrochemical sensor module with at least a first calibration and/or quality control reagent and second calibration and/or quality control reagent to obtain first and second values, wherein the first and second calibration and/or quality control reagents have pCO₂ concentrations that are substantially the same;
contacting the electrochemical sensor module with at least the first calibration and/or quality control reagent and a third calibration and/or quality control reagent to obtain third and fourth values, wherein the first and third calibration and/or quality control reagents have substantially the same pH;
determining a pH slope (pH) for the pH sensor of the electrochemical sensor module based on the first and second values obtained with the at least first and second calibration and/or quality control reagents;
determining a pH (HCO₃/pCO₂) slope for the HCO₃ sensor of the electrochemical sensor module based on the third and fourth values obtained with the at least first and third calibration and/or quality control reagents;
calculating a ΔpH slope by subtracting the pH (HCO₃/pCO₂) slope from the pH (pH) slope; and
determining that a micro clot is present on the pH sensor when the ΔpH slope is above a threshold value.

15. The method of claim 14, further comprising the steps of:
generating a result indicating that the pH sensor result for a sample is questioned when the ΔpH slope is above the threshold value; and
performing a clot removal step when the ΔpH slope is above the threshold value.

16. The method of claim 14, wherein each of the first and second calibration and/or quality control reagents has a pCO₂ concentration of about 35 mmHg, and the third calibration and/or quality control reagent has a pCO₂ concentration of about 70 mmHg, and wherein each of the first and third calibration and/or quality control reagents have a pH of about 7.4, and the second calibration and/or quality control reagent has a pH of about 6.8.
